(19) United States
(12) Patent Application Publication
(10) Pub. No.: US 2023/0218692 A1
(43) Pub. Date: Jul. 13, 2023

(54) METHOD FOR PRODUCING A PURIFIED CANNABACEAE BIOMASS EXTRACT

(71) Applicant: PurCann Pharma inc., Québec (CA)

(72) Inventors: Xavier PIGEON, Québec (CA); Claudia CARPENTIER, Québec (CA); Maëlle DERRIEN, Québec (CA); Simon GIRET, Québec (CA)

(21) Appl. No.: 17/996,548

(22) PCT Filed: Apr. 13, 2021
(86) PCT No.: PCT/CA2021/050492

§ 371 Date: Oct. 19, 2022

Related U.S. Application Data
(60) Provisional application No. 63/018,096, filed on Apr. 30, 2020.

(51) Int. Cl. A61K 36/185; A61K 31/05; A61K 31/352

(52) U.S. Cl. CPC A61K 36/185; A61K 31/05; A61K 31/352; A61K 2236/333; A61K 2236/53; A61K 2236/37; A61K 2236/17; A61K 2236/15

(57) ABSTRACT

The present disclosure relates to a method for producing a purified cannabaceae biomass extract, wherein a crude alcoholic cannabaceae biomass extract comprising at least two cannabinoid compounds in an alcoholic solvent is circulating through a powdered layer of activated carbon and the purified cannabaceae biomass extract is recovered with a ratio of cannabinoid compounds in the purified cannabaceae biomass extract is higher than the ratio of the cannabinoid compounds in the crude alcoholic cannabaceae biomass extract.

### CROSS-REFERENCE TO RELATED APPLICATION

The present application is claiming priority from U.S. Provisional Application No. 63/018,096 filed Apr. 30, 2020, the content of which is hereby incorporated by reference in its entirety.

### FIELD OF THE DISCLOSURE

The present disclosure relates to a method for producing a purified cannabaceae biomass extract.

### BACKGROUND

The cannabinoid compounds have been studied for various reasons over the past decades. In the recent years, the use of cannabinoids as pharmaceutical, cosmetic or recreational products has increased in several countries. Purified cannabinoids, especially cannabinoids without tetrahydrocannabinol are particularly sought after to avoid psychoactive effects.

Currently, the major sources of cannabinoids are coming from the extraction of hemp and/or cannabis biomass. However, due to the nature of biomass used, cannabinoids are mixed together in the extract in different proportions and it is a challenge to separate them from each other.

### SUMMARY OF THE DISCLOSURE

An aspect relates to a method for producing a purified cannabaceae biomass extract, the method comprising providing a crude alcoholic cannabaceae biomass extract comprising at least two cannabinoid compounds in an alcoholic solvent; circulating said crude alcoholic cannabaceae biomass extract through a powdered layer of activated carbon; and recovering said purified cannabaceae biomass extract, wherein a ratio of a first cannabinoid compound to a second cannabinoid compound in said purified cannabaceae biomass extract is higher than a ratio of said first cannabinoid compound to said second cannabinoid compound in said crude alcoholic cannabaceae biomass extract.

In an embodiment, the cannabaceae biomass is at least one of hemp and cannabis.

In a further embodiment, the cannabaceae biomass is at least one of hemp, Cannabis Sativa, Cannabis Indica, a hybrid of Cannabis Sativa and Cannabis Indica and Cannabis ruderalis.

In another embodiment, the at least two cannabinoid compounds comprise at least one of tetrahydrocannabinol (THC), cannabichromene (CBC), cannabidiol (CBD), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL) sesquicannabigerol, cannabicoumarononic acid, cannabinoid benzoquinone, and cannabispiroindane.

In an embodiment, the at least two cannabinoid compounds is at least one of THC and Tetrahydrocannabinolic acid (THCA), and at least one CBD and cannabidiolic acid (CBDA).

In another embodiment, the at least two cannabinoid compounds comprise a first cannabinoid compound consisting of CBD, CBDA or a combination thereof, and second cannabinoid compound consisting of THC, THCA or a combination thereof.

In a further embodiment, the cannabaceae biomass is pre-treating the before the step of extracting the biomass.

In another embodiment, the cannabaceae biomass is dried before the step of extracting the biomass.

In a particular embodiment, the pre-treating step comprises grinding the cannabaceae biomass.

In an embodiment, the cannabaceae biomass is shredded to pieces of from about 1 to about 0,1-inch-long.

In an embodiment, the pre-treating step comprises grinding and drying the cannabaceae biomass.

In an embodiment, the drying step reduces moisture of the cannabaceae biomass to less than about 15% (wt/wt ratio).

In a further embodiment, the step of providing a crude cannabaceae biomass extract comprises extracting the biomass, using supercritical CO₂ extraction, an alkane solvent, or an alcoholic solvent.

In an embodiment, the alkane solvent is at least one of propane, butane, isobutane, pentane, isopentane, neopentane, hexane, 2-methylpentane, 3-methylpentane, 2,2-dimethylbutane, 2,3-dimethylbutane, heptane, methylhexane and dimethylpentane.

In another embodiment, the biomass is extracted in the alkane solvent at ratio of alkane solvent/cannabaceae biomass from about 5:1 to about 20:1.

In an embodiment, the alcoholic solvent is ethanol.

In a further embodiment, the biomass is extracted in the alcoholic solvent at a ratio of alcoholic solvent/cannabaceae biomass from about 5:1 to about 20:1.

In a further embodiment the biomass is extracted at a temperature of from about −80° C. to about 30° C.

In another embodiment, the total amount of cannabinoid compounds in the biomass extract is comprised between 15 and 60% by weight.

In an embodiment, the step of providing a crude cannabaceae biomass extract comprises providing a crude alcoholic cannabaceae biomass extract comprising evaporating the alkane solvents of the crude cannabaceae biomass extract to provide a crude evaporated extract and diluting the crude evaporated extract in the alcoholic solvent to produce the crude alcoholic cannabaceae biomass extract.

In another embodiment, the step of providing the crude alcoholic cannabaceae biomass extract comprises removing lipids by winterization to provide a crude winterized alcoholic cannabaceae biomass extract.

In an embodiment, the crude winterized alcoholic cannabaceae biomass extract is further passed through a powdered layer of activated carbon at a temperature of from about 10° C. to about 40° C.

In a supplemental embodiment, the cannabaceae biomass extract comprises a mass ratio of CBD+CBDA/THC+THCA of at least about 5.

In an embodiment, the powdered activated carbon is from wood carbon, coal carbon, or biochar.

In an embodiment, the powdered activated carbon is acid-activated wood carbon, steam-activated coal carbon, or activated carbon from biochar.

In another embodiment, the activated carbon powder is immobilized onto an inert support.

In an embodiment, the powdered activated carbon is at least packed by gravity or by a mechanical packing force.

In an embodiment, the powdered activated carbon is SiliaCarb™ E-PAK® HA activated Carbons. The method as defined herein, wherein the crude alcoholic cannabaceae biomass extract circulated through the powdered layer of activated carbon comprises about lg of solubilized solid compounds for at least about 1.5 g of said activated carbon.

In another embodiment, the crude alcoholic cannabaceae biomass extract circulated through the powdered layer of activated carbon comprises about 1 g of solubilized solid compounds for from about 1.5 g to about 4 g of said activated carbon.

In another embodiment, the residence time of the crude alcoholic cannabaceae biomass extract on the activated carbon is between about 0.5 and about 5 minutes.

In an embodiment, further comprising the recovered purified cannabaceae biomass extract is solvent-evaporated to provide an evaporated purified cannabaceae biomass extract.

In an embodiment, the content of THC and THCA relative to the content of CBD and CBDA in the purified cannabaceae biomass extract is lower than in the crude cannabaceae biomass extract.

In another embodiment, the concentration ratio of CBD+CBDA to THC+THCA in the purified cannabaceae biomass extract is 3 to 20 time higher than in the crude alcoholic cannabaceae biomass extract.

In another embodiment, the content of THC and THCA is not detectable or quantifiable in the purified cannabaceae biomass extract.

### BRIEF DESCRIPTION OF THE DRAWINGS

Reference will now be made to the accompanying drawings.

FIG. 1 illustrates a flow chart of the process of producing a purified cannabaceae biomass extract in accordance to an embodiment.

### DETAILED DESCRIPTION

The present disclosure relates to a method for producing a purified cannabaceae biomass extract.

It has been discovered that cannabinoid compounds from a cannabaceae biomass extract (such as hemp and/or cannabis) may be separated using activated carbon.

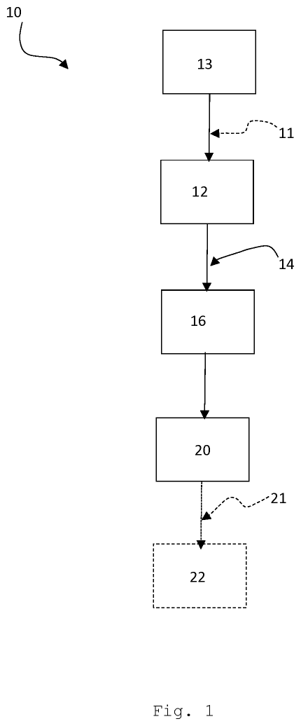

As seen in FIG. 1, it is provided a method for producing a purified cannabaceae biomass extract 20, the method 10 comprising providing a crude alcoholic cannabaceae biomass extract 12 comprising at least two cannabinoid compounds in an alcoholic solvent; circulating 14 the crude alcoholic cannabaceae biomass extract through a powdered layer of activated carbon 16; and recovering the purified cannabaceae biomass extract 20, wherein a ratio of a first cannabinoid compound to a second cannabinoid compound in the purified cannabaceae biomass extract is higher than a ratio of the first cannabinoid compound to the second cannabinoid compound in the crude alcoholic cannabaceae biomass extract.

In one embodiment, the cannabaceae biomass is hemp and/or cannabis, preferably at least one of hemp, Cannabis Sativa, Cannabis Indica, a hybrid of Cannabis Sativa and Cannabis Indica and Cannabis ruderalis, and more preferably hemp. The cannabaceae biomass may be obtained from any part of the cannabaceae plant.

In one embodiment, the at least one cannabinoid compound is at least one of tetrahydrocannabinol (THC) (such as Δ9-THC, Δ8-THC), cannabichromene (CBC), cannabidiol (CBD), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL) and other types (such sesquicannabigerol; cannabicoumarononic acid; cannabinoid benzoquinone; cannabispiroindane).

In one embodiment, said at least two cannabinoid compounds is at least one of THC and Tetrahydrocannabinolic acid (THCA), and at least one CBD and cannabidiolic acid (CBDA).

In one embodiment, said first cannabinoid compound is CBD, CBDA or a combination thereof.

In one embodiment, said second cannabinoid compound is THC, THCA or a combination thereof.

The structures of four representative compounds are shown in the following table:

| | |
|---|---|
| CBDA (cannabidiolic acid) | 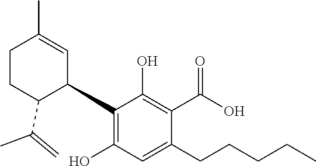 [SMILES: C=C(C)[C@@H]1CCC(C)=C[C@H]1c1c(O)cc(CCCCC)c(C(=O)O)c1O] |
| Cannabidiol (CBD) | 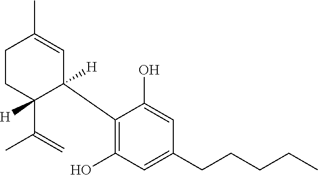 [SMILES: C=C(C)[C@@H]1CCC(C)=C[C@H]1c1c(O)cc(CCCCC)cc1O] |
| Tetrahydrocannabinolic acid (THCA) | 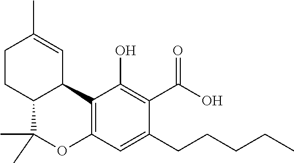 [SMILES: CCCCCc1cc2c(c(O)c1C(=O)O)[C@@H]1C=C(C)CC[C@H]1C(C)(C)O2] |
| Tetrahydrocannabinol (THC) | 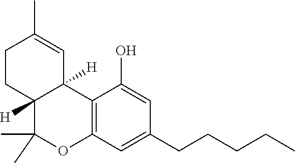 [SMILES: CCCCCc1cc(O)c2c(c1)OC(C)(C)[C@@H]1CCC(C)=C[C@@H]21] |

In one embodiment, the method is comprising a step of pre-treating 11 the cannabaceae biomass 13 before the step of extracting the biomass.

In one embodiment, said pre-treating step 11 is comprising drying the cannabaceae biomass 13.

In one embodiment, said pre-treating step 11 is comprising grinding the cannabaceae biomass 13. The grinding step is causing a size reduction of the biomass. Examples include shredding of the cannabaceae biomass to pieces of from about 1 to about 0,1-inch-long, preferably about 0,2 inch long.

In one embodiment, said pre-treating step 11 is comprising grinding and drying the cannabaceae biomass 13.

In one embodiment the drying step is reducing moisture of the cannabaceae biomass to less than about 15% (wt/wt ratio).

In one embodiment, the step of providing a crude cannabaceae biomass extract 12 is comprising extracting the biomass, preferably hemp and/or cannabis, using supercritical CO₂ extraction. The CO₂ extraction has been previously reported and is known to the skilled person. A process is disclosed for example in U.S. Pat. No. 6,403,126 B1, which is incorporated herein by reference.

In one embodiment, the step of providing a crude cannabaceae biomass extract 12 is comprising extracting the biomass, preferably hemp and/or cannabis, using an alkane solvent. Non-limiting examples include, propane, butane, isobutane, pentane, isopentane, neopentane, hexane, 2-methylpentane, 3-methylpentane, 2,2-dimethylbutane, 2,3-dimethylbutane, heptane, methylhexane (2 isomers) or dimethylpentane (4 isomers). Of those, alkane solvents with lower boiling points are preferred. The alkane extraction has been previously reported and is known to the skilled person. A process is disclosed for example in U.S. Pat. No. 6,403,126 B1, which is incorporated herein by reference.

A typical ratio of alkane solvent/cannabaceae biomass is from about 5:1 to about 20:1.

In one embodiment the step of providing a crude alcoholic cannabaceae biomass extract 12 is comprising extracting the biomass, preferably hemp and/or cannabis, using an alcoholic solvent, preferably ethanol, such as 95% and more ethanol.

A typical ratio of alcoholic solvent/cannabaceae biomass is from about 5:1 to about 20:1.

In one embodiment, the step of providing a crude cannabaceae biomass extract or crude alcoholic cannabaceae biomass extract 12 using an alkane or alcoholic solvent is conducted at a temperature of from about −80° C. to about 30° C. or from about 20° C. to about 30° C. or for an alcoholic solvent, such as ethanol, above the melting point or preferably from about 0° C. to about 30° C. or from about 20° C. to about 30° C.

In one embodiment, the total amount of cannabinoid compounds in the evaporated crude cannabaceae biomass extract or evaporated crude alcoholic cannabaceae biomass extract is comprised between 15 and 60% by weight, when assessed by HPLC by the method of De Backer, B. et al, J. chromatogr. B (2009) on a standard curve between 5 and 100 ppm in ethanol.

In one embodiment, the step of providing the crude alcoholic cannabaceae biomass extract 12 is comprising evaporating the alkane solvents of the crude cannabaceae biomass extract to provide a crude evaporated extract and diluting the crude evaporated extract in the alcoholic solvent (such as ethanol, including 95% or more) to produce the crude alcoholic cannabaceae biomass extract.

In one embodiment, the step of providing the crude alcoholic cannabaceae biomass extract 12 is comprising removing lipids (winterization/winterizing) to provide a crude winterized alcoholic cannabaceae biomass extract. Winterization is known in the art and can commonly be done by one of the cold ethanol removal, (i.e. by cooling the alcoholic extract sufficiently to cause the wax to solidify and be removed), BHO (Butane Hash Oil) extraction or CO₂ extraction.

In one embodiment, the step of circulating 14 said crude alcoholic cannabaceae biomass extract or said crude winterized alcoholic cannabaceae biomass extract 12 through a powdered layer of activated carbon 16 is conducted at a temperature of from about 10° C. to about 40° C. or about 20° C. to about 30° C.

In one embodiment, the cannabaceae biomass, the crude cannabaceae biomass extract, the evaporated crude cannabaceae biomass extract, the crude alcoholic cannabaceae biomass extract, or the evaporated crude alcoholic cannabaceae biomass extract is comprising a mass ratio of CBD+CBDA/THC+THCA of at least about 5, preferably between about 5 and about 100.

In one embodiment, the powdered activated carbon may be acid-activated (wood) carbon, steam-activated (coal) carbon. Preferably, the activated carbon powder is immobilized onto an inert support. In one embodiment, the powdered activated carbon is at least packed by gravity, preferably by a mechanical packing force. An example of commercially available powdered activated carbon immobilized onto a support includes such as SiliaCarb™ E-PAK® HA activated Carbons. Although the SiliaCarb™ E-PAK® carbon filters have generally been used at room temperature for the removal of sub-micron sized particulates in water or other liquids, they have been found to be suitable for use in the method described herein.

In one embodiment of said step of circulating 14 said crude alcoholic cannabaceae biomass extract 12 through a powdered layer of activated carbon 16, said crude alcoholic cannabaceae biomass extract is comprising about 1 g of solubilized solid compounds for at least about 1.5 g of said activated carbon, or from about 1.5 g to about 4 g of said activated carbon.

In one embodiment, the residence time of the crude alcoholic cannabaceae biomass extract on the activated carbon is between about 0.5 and about 5 minutes. The flow rate of the crude alcoholic cannabaceae biomass extract on the activated carbon is between about 0.5 and about 2.5 BV (bed volume) per minute.

In one embodiment, the recovered purified cannabaceae biomass extract 20 is solvent-evaporated 21 to provide an evaporated purified cannabaceae biomass extract 22.

In one embodiment, the content of THC and THCA relative to the content of CBD and CBDA in the purified cannabaceae biomass extract is lower than in the crude cannabaceae biomass extract. In one embodiment, the concentration ratio of CBD+CBDA to THC+THCA in the purified cannabaceae biomass extract is 3 to 20 time higher than in the crude alcoholic cannabaceae biomass extract. In one embodiment, the content of THC and THCA is not detectable or quantifiable in the purified cannabaceae biomass extract (i.e. before alcohol (preferably ethanol) evaporation) when assessed by the method of De Backer, B. et al, J. chromatogr. B (2009) described herein.

### EXAMPLES

The following reagents, equipment and conditions were used.

Ethanol 190 proof, food grade was obtained from Greenfield. Acetonitrile 100% HPLC grade, ammonium formate, formic acid, And MEOH 100% HPLC grade were obtained from Sigma. Cannabinoid standards (THC, THCA, CBN, CBD, CBDA) were obtained from Cerrilant.

SiliaCarb™ E-pak cartridges and the screening kit were obtained from SiliCycle inc. The alcoholic cannabaceae biomass extract is circulated through the SiliaCarb™ E-PAK using a Buchi Module C-601 Pump.

The cannabinoid biomass/plants were stored in polyethylene, Zyploc®-type bags.

All chromatographic runs were carried out using Agilent 1100 HPLC Series, consisting of a G1322A solvent degasser, a G1311A quaternary solvent pump, a G1313A autosampler, a G1316A column compartment and a G1315B photodiode-array detector. For this study the detector was set to 230 nm. Chromatographic separations were performed using a SiliaChrom dt C18 3 μm 4.6×150 mm. Column temperature was set to 35° C. and the flow was set to 1.5 mL/min. The analytical method was based on De Backer, B.; et al Innovative Development and Validation of an HPLC/DAD Method for the Qualitative and Quantitative Determination of Major Cannabinoids in Cannabis Plant Material. J. chromatogr. B (2009), 877, pp 4115-4124, the content of which is incorporated herein by reference. In summary, samples were prepared by dilution in 100% HPLC grade MeOH, to a concentration expected to be near 50 ppm (i.e. center of the standard calibration curve), The injection volume was 10 μL and the sample was eluted under isocratic condition with 75/25 (v/v) acetonitrile/ammonium formate 50 mM+0.1% (v/v) formic acid.

### Example 1

Hemp plants, cultivar CANMA. All the plants were collected and stored in a bag after flash drying. Flash drying consisted of a biomass drying at high temperature (i.e. 600-800° C.) during a short period of time (5-10 seconds) to produce biomass having 10-20% humidity. In this example, the biomass was at 15% humidity. Ethanol 190 proof, food grade was added to the non-grinded plant biomass (size of biomass chunk comprised between 2 cm and 0.1 mm) in an ethanol/biomass ratio of about 15:1 (weight basis) and agitated at 100 rpm at −20° C. for 15 min and then filtered on a Buchner filter. The crude ethanolic hemp extract comprised solubilized solids compounds (concentration of 4.89 mg/mL.

SiliaCarb™ E-PAK® HA Cartridges Activated Carbon, 5×1 cm (5 g of activated carbon) were use with a Laboratory Scale SiliaCarb™ E-PAK® Cartridges housing kit. The mass ratio of activated carbon/total solubilized solids compounds in the crude ethanolic hemp extract was 2.92. The flow rate of crude ethanolic hemp extract was set at 0.66 BV/minute. 350 mL of the crude ethanolic hemp extract was passed on the column and the purified hemp extract was collected, evaporated and analysed by HPLC.

The results of the cannabinoid compounds analysis are summarized in Table 1.

TABLE 1

| | Concentration of | Concentration of |
|---|---|---|
| | compounds in | compounds in |
| | evaporated crude | evaporated purified |
| | ethanolic hemp extract | hemp extract |
| Total CBD | 27.55 | 45.00 |
| (weight %) | | |
| Total THC | 2.02 | N. Q. |
| (weight %) | | |

N. Q.: lower than quantification limit

### Example 2

Hemp plants, cultivar CANMA. All the plants were collected and stored in a bag after flash drying. Flash drying consisted of a biomass drying at high temperature (i.e. 600-800° C.) during a short period of time (5-10 seconds) to produce biomass having 10-20% humidity. In this example, the biomass was at 15% humidity. Ethanol 190 proof, food grade was added to the non-grinded plant biomass (size of biomass chunk comprised between 2 cm and 0.1 mm) in an ethanol/biomass ratio of about 15:1 (weight basis) and agitated at 100 rpm at room temperature for 15 min and then filtered on a Buchner filter. The crude ethanolic hemp extract comprised solubilized solids compounds concentration of 7.95 mg/mL.

SiliaCarb™ E-PAK® HA Cartridges Activated Carbon, 5×1 cm (5 g of carbon) were use with a Laboratory Scale E-PAK® Cartridges housing kit. The mass ratio of activated carbon/total solubilized solids compounds in the crude ethanolic hemp extract was 1.80. The flow rate of crude ethanolic hemp extract was set at 0.66 BV/minute. 350 mL of the crude ethanolic hemp extract was passed on the column and the purified hemp extract was collected, evaporated and analysed by HPLC.

The results of the cannabinoid compounds are summarized in Table 2.

TABLE 2

| | Concentration of | Concentration of |
|---|---|---|
| | compounds in | compounds in |
| | evaporated crude | evaporated purified |
| | ethanolic hemp extract | hemp extract |
| Total CBD | 20.65 | 29.51 |
| (weight %) | | |
| Total THC | 1.93 | 0.66 |
| (weight %) | | |

### Example 3

Hemp plants, cultivar CANMA. All the plants were collected and stored in a bag after flash drying. Flash drying consisted of a biomass drying at high temperature (i.e. 600-800° C.) during a short period of time (5-10 seconds) to produce biomass having 10-20% humidity. In this example, the biomass was at 15% humidity. Ethanol 190 proof, food grade was added to the non-grinded plant biomass (size of biomass chunk comprised between 2 cm and 0.1 mm) in an ethanol/biomass ratio of about 15:1 (weight basis) and agitated at 100 rpm at 0° C. for 15 min and then filtered on a Buchner filter. The crude ethanolic hemp extract comprised solubilized solids concentration of 7.32 mg/mL.

SiliaCarb™ E-PAK® HA Cartridges Activated Carbon, 5×1 cm (5 g of activated carbon) were use with a Laboratory Scale E-PAK® Cartridges housing kit. The mass ratio of activated carbon/total solubilized solid compounds in the crude ethanolic hemp extract was 1.95. The flow rate of crude ethanolic hemp extract was set at 0.66 BV/minute. 350 mL of crude ethanolic hemp extract was passed on the column and the purified hemp extract was collected, evaporated and analysed by HPLC.

The results of the cannabinoid compounds analysis are summarized in Table 3.

TABLE 3

| | Concentration of | Concentration of |
|---|---|---|
| | compounds in | compounds in |
| | evaporated crude | evaporated purified |
| | ethanolic hemp extract | hemp extract |
| Total CBD | 17.71 | 25.74 |
| (weight %) | | |
| Total THC | 2.08 | N. Q. |
| (weight %) | | |

N. Q.: lower than quantification limit

### Example 4

Hemp plants, cultivar JOEY. All the plants were collected and stored in a bag after drying. Drying consisted of a biomass drying at room temperature (i.e. 25° C.) during a long period of time (1-3 weeks), in a well-ventilated room, to produce biomass having 7-15% humidity. In this example, the biomass was at 10% humidity. The biomass was placed in filtration bag and cold ethanol 190 proof (−40° C.), food grade was added to the non-grinded plant biomass (size of biomass chunk comprised between 2 cm and 0.1 mm) in an ethanol/ biomass ratio of about 15:1 (weight basis) and agitated with a cup-30 centrifuge from Delta Separation®. The agitation was set between 85-130 rpm for 5 min then the crude ethanolic hemp extract was centrifuged out through the filtration bag by centrifugation at 1500 RPM for 5 min. The crude ethanolic hemp extract comprised an CBD total to THC total ratio equal to 27.

SiliaCarb™ E-PAK® HA Cartridges Activated Carbon, 16.5×100 cm (6.8 kg of activated carbon C947 Carbon) were use with an industrial Scale E-PAK® Cartridges housing kit. The mass ratio of activated carbon/total solubilized solid compounds in the crude ethanolic hemp extract was estimate at 2.75 for the targeted fraction. The flow rate of crude ethanolic hemp extract was set at 0.5 BV/minute. The crude ethanolic hemp extract was passed on the column and the purified hemp extract was collected, evaporated and analysed by HPLC.

The results of the cannabinoid ratio of CBD total to THC total for the corresponding fraction was 100.

While the description has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

1. A method for producing a purified cannabaceae biomass extract, the method comprising:
providing a crude alcoholic cannabaceae biomass extract comprising at least two cannabinoid compounds in an alcoholic solvent;
circulating said crude alcoholic cannabaceae biomass extract through a powdered layer of activated carbon; and
recovering said purified cannabaceae biomass extract, wherein a ratio of a first cannabinoid compound to a second cannabinoid compound in said purified cannabaceae biomass extract is higher than a ratio of said first cannabinoid compound to said second cannabinoid compound in said crude alcoholic cannabaceae biomass extract.

2. The method of claim 1, wherein the cannabaceae biomass is at least one of hemp and cannabis.

3. The method of claim 1, wherein the cannabaceae biomass is at least one of hemp, Cannabis Sativa, Cannabis Indica, a hybrid of Cannabis Sativa and Cannabis Indica and Cannabis ruderalis.

4. The method of claim 1, wherein the at least two cannabinoid compounds comprise at least one of tetrahydrocannabinol (THC), cannabichromene (CBC), cannabidiol (CBD), cannabigerol (CBG), cannabinol (CBN), cannabinodiol (CBND), cannabicyclol (CBL), cannabielsoin (CBE), cannabicitran type (CBCT), cannabitriol (CBT), cannabielsoin (CBL) sesquicannabigerol, cannabicoumarononic acid, cannabinoid benzoquinone, and cannabispiroindane.

5. (canceled)

6. The method of claim 4, wherein said at least two cannabinoid compounds comprise a first cannabinoid compound consisting of CBD, CBDA or a combination thereof, and second cannabinoid compound consisting of THC, THCA or a combination thereof.

7. (canceled)

8. The method of claim 1, wherein the cannabaceae biomass is dried before the step of extracting the biomass.

9. The method of claim 1, further comprising grinding the cannabaceae biomass.

10. The method of claim 9, wherein the cannabaceae biomass is shredded to pieces of from about 1 to about 0,1-inch-long.

11-12. (canceled)

13. The method of claim 1, wherein the step of providing a crude cannabaceae biomass extract comprises extracting the biomass, using supercritical CO₂ extraction, an alkane solvent, or an alcoholic solvent.

14. The method of claim 13, wherein the alkane solvent is at least one of propane, butane, isobutane, pentane, isopentane, neopentane, hexane, 2-methylpentane, 3-methylpentane, 2,2-dimethylbutane, 2,3-dimethylbutane, heptane, methylhexane and dimethylpentane.

15. The method of claim 13, wherein the biomass is extracted in the alkane solvent at ratio of alkane solvent/cannabaceae biomass from about 5:1 to about 20:1.

16-18. (canceled)

19. The method of claim 13, wherein the total amount of cannabinoid compounds in the biomass extract is comprised between 15 and 60% by weight.

20. The method of claim 13, wherein the step of providing a crude cannabaceae biomass extract comprises providing a crude alcoholic cannabaceae biomass extract comprising evaporating the alkane solvents of the crude cannabaceae biomass extract to provide a crude evaporated extract and diluting the crude evaporated extract in the alcoholic solvent to produce the crude alcoholic cannabaceae biomass extract.

21. The method of claim 20, wherein the step of providing the crude alcoholic cannabaceae biomass extract comprises removing lipids by winterization to provide a crude winterized alcoholic cannabaceae biomass extract.

22. (canceled)

23. The method of claim 1, wherein the cannabaceae biomass extract comprises a mass ratio of CBD+CBDA/THC+THCA of at least about 5.

24. The method of claim 1, wherein the powdered activated carbon is from wood carbon, coal carbon, or biochar.

25. The method of claim 1, wherein the powdered activated carbon is acid-activated wood carbon, steam-activated coal carbon, or activated carbon from biochar.

26-28. (canceled)

29. The method of claim 1, wherein the crude alcoholic cannabaceae biomass extract circulated through the powdered layer of activated carbon comprises about 1 g of solubilized solid compounds for at least about 1.5 g of said activated carbon.

30-32. (canceled)

33. The method of claim 1, wherein the content of THC and THCA relative to the content of CBD and CBDA in the purified cannabaceae biomass extract is lower than in the crude cannabaceae biomass extract, or wherein the content of THC and THCA is not detectable or quantifiable in the purified cannabaceae biomass extract.

34. The method of claim 1, wherein the concentration ratio of CBD+CBDA to THC+THCA in the purified cannabaceae biomass extract is 3 to 20 time higher than in the crude alcoholic cannabaceae biomass extract.

35. (canceled)
